(19) United States
(12) Patent Application Publication
(10) Pub. No.: US 2008/0213857 A1
(43) Pub. Date: Sep. 4, 2008

(54) Patterning substrate and cell culture substrate

(75) Inventors: Hideyuki Miyake, Tokyo (JP); Hideshi Hattori, Tokyo (JP); Hironori Kobayashi, Tokyo (JP)

Correspondence Address: LADAS & PARRY LLP, 224 SOUTH MICHIGAN AVENUE, SUITE 1600, CHICAGO, IL, 60604, US

(73) Assignee: Dai Nippon Printing Co., Ltd.

(21) Appl. No.: 12/002,863

(22) Filed: Dec. 19, 2007

Related U.S. Application Data
(62) Division of application No. 11/044,437, filed on Jan. 27, 2005.

(30) Foreign Application Priority Data
Jan. 28, 2004 (JP) 2004-020250

(51) Int. Cl. C12N 11/14

(52) U.S. Cl. 435/176

(57) ABSTRACT

The present invention intends primarily to provide such as a cell culture patterning substrate that is used to adhere cells in a highly precise pattern on a base material to culture and a cell culture substrate on which cells is adhered in a highly precise pattern.

In order to attain the object, the invention provides a patterning substrate comprising a base material, a photocatalyst-containing layer which is formed on the base material and comprises at least a photocatalyst, and a cell adhesion-inhibiting layer which is formed on the photocatalyst-containing layer and comprises a cell adhesion-inhibiting material that has cell adhesion-inhibiting properties of inhibiting adhesion to cells and is decomposed or denatured by action of the photocatalyst on the basis of irradiation with energy.

### BACKGROUND OF THE INVENTION

1. Field of the Invention

The present invention relates to a cell culture patterning substrate capable of adhering cells in a highly precise pattern, a patterning substrate used for forming the cell culture patterning substrate, a coating liquid for patterning substrate used for forming the patterning substrate, and a cell culture substrate on which cells are adhered in a highly precise pattern.

2. Description of the Related Art

At present, cell cultures of various animals and plants are performed, and also new cell culture methods are in development. The technologies of the cell culture are utilized, such as, to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culturing plate with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method of arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in Japanese Patent Application Laid-Open (JP-A) No. 2-245181, an electric charge-retaining medium on which an electrostatic pattern is formed is applied to culture cells for the purpose of proliferating nerve cells in a form of circuit, and the like. Furthermore, JP-A No. 3-7576 tries to arrange cultured cells on a surface on which a cell non-adhesive or cell adhesive photosensitive hydrophilic polymer has been patterned by a photolithography method.

Furthermore, JP-A No. 5-176753 discloses a cell culture base material on which a substance such as collagen and the like affecting on the adhesion ratio and form of cells is patterned, and a method of producing this base material by a photolithography method. By culturing cells on such a base material, a larger amount of cells can be adhered on a surface on which collagen or the like is patterned, to realize patterning of cells.

However, such patterning of cell culture regions may be required to be highly precise depending on applications. In the case of conducting patterning by a photolithography method using a photosensitive material as described above, a highly precise pattern can be obtained; however, a cell adhesive material is required to have photosensitivity, and it is difficult in many cases to conduct chemical modification to impart such photosensitivity to, for instance, biopolymers and the like; thereby leading to extremely narrow width in selectivity of cell adhesive materials, problematically. Furthermore, in a photolithography method using a photo resist, it is necessary to use a liquid developer and the like, and these can affect adversely in culturing cells in some cases.

Furthermore, as a method of forming a highly precise pattern of a cell adhesive material, a Micro Contact Printing method is proposed by George M. Whitesides, Harvard University (for example, U.S. Pat. Nos. 5,512,131 and 5,900,160, JP-A Nos. 9-240125 and 10-12545 etc). However, there is a problem in that it is difficult to industrially produce a cell culture base material having a pattern of a cell adhesive material using this method.

### SUMMARY OF THE INVENTION

In this connection, it is desired to provide such as a cell culture patterning substrate that is used to cause cells to adhere in highly precise pattern on to a base material to culture, and a cell culture substrate to which cells are adhered in highly precise pattern.

The present invention provides a patterning substrate comprising a base material, a photocatalyst-containing layer which is formed on the base material and comprises at least a photocatalyst, and a cell adhesion-inhibiting layer which is formed on the photocatalyst-containing layer and comprises a cell adhesion-inhibiting material that has cell adhesion-inhibiting properties of inhibiting adhesion to cells and is decomposed or denatured by action of the photocatalyst on a basis of irradiation with energy.

Since the cell adhesion-inhibiting layer is formed on the photocatalyst-containing layer according to the invention, the irradiation of the patterning substrate with energy makes it possible to decompose or denature the cell adhesion-inhibiting material contained in the cell adhesion-inhibiting layer to make the cell adhesive properties good. The region not irradiated with the energy, by the cell adhesion-inhibiting material, can be rendered a region low in cell adhesive properties. It is therefore possible to render the patterning substrate a patterning substrate capable of forming easily a region good in cell adhesive properties, where the cell adhesion-inhibiting material is decomposed or denatured, and a region low in cell adhesive properties, which contains the cell adhesion-inhibiting material, without requiring any special device or complicated step.

In the invention, it is preferred that the cell adhesion-inhibiting layer comprises a cell adhesive material having cell adhesive properties at least after the material is irradiated with the energy. This makes it possible to make better the cell adhesive properties of the region where the cell adhesion-inhibiting material is decomposed or denatured.

In the invention, a light-shielding portion may be formed on the base material or the photocatalyst-containing layer. In the case of irradiating energy onto the entire surface of the patterning substrate from the side of the base material thereof, only the cell adhesion-inhibiting material comprised in the cell adhesion-inhibiting layer outside the region where the light-shielding portion is formed can be decomposed or denatured without decomposing or denaturing the cell adhesion-inhibiting material in the cell adhesion-inhibiting layer on the region where the light-shielding portion is formed.

The invention also provides a cell culture patterning substrate, wherein the cell adhesion-inhibiting layer of any one of the above-mentioned patterning substrates comprises a cell adhesion portion where the cell adhesion-inhibiting material is decomposed or denatured in a pattern form, and a cell adhesion-inhibiting portion which is a region other than the cell adhesion portion.

According to the invention, the cell culture patterning substrate comprises the cell adhesion portion good in cell adhesive properties, where the cell adhesion-inhibiting material is decomposed or denatured, and the cell adhesion-inhibiting portion low in cell adhesive properties, where the cell adhesion-inhibiting material is not decomposed or denatured; therefore, the cell culture patterning substrate can be rendered a cell culture patterning substrate capable of causing cells to adhere highly precisely only onto the cell adhesion portion without requiring any complicated step, any treating solution or the like that produces a bad effect on the cells.

The present invention also provides a cell culture substrate, wherein the cells adhere onto the cell adhesion portion of the above-mentioned cell culture patterning substrate.

According to the invention, the use of the cell culture patterning substrate having the above-mentioned cell adhesion portion and cell adhesion-inhibiting portion makes it possible that cells are caused to adhere easily onto the cell adhesion portion.

The present invention also provides a coating liquid for patterning substrate comprising a cell adhesion-inhibiting material which has cell adhesion-inhibiting properties of inhibiting adhesion to cells and is decomposed or denatured by action of a photocatalyst on a basis of irradiation with energy, and a cell adhesive material which has cell adhesive properties at least after the material is irradiated with the energy.

According to the invention, by applying the coating liquid for patterning substrate on, for example, a layer or the like that contains a photocatalyst and then irradiating energy thereto, the cell adhesion-inhibiting material can easily be decomposed to render the region irradiated with the energy a region having cell adhesive properties. Since the region irradiated with the energy contains the cell adhesive material, the region can be rendered a region better in cell adhesive properties. On the other hand, in the region not irradiated with the energy, the region is hindered from adhering onto cells by the cell adhesion-inhibiting material; therefore, the region can be rendered a region low in cell adhesive properties. Accordingly, the coating liquid for patterning substrate is a coating liquid for patterning substrate capable of forming easily a region good in cell adhesive properties and a region low in cell adhesive properties.

### BRIEF DESCRIPTION OF THE DRAWINGS

FIG. 1 is a schematic sectional view showing an example of the patterning substrate of the present invention.

FIG. 2 is a schematic sectional view showing another example of the patterning substrate of the invention.

FIG. 3 is a schematic sectional view showing still another example of the patterning substrate of the invention.

FIG. 4 is a schematic sectional view showing an example of the cell culture patterning substrate of the invention.

FIG. 5 is a schematic sectional view showing another example of the cell culture patterning substrate of the invention.

FIGS. 6A to 6C are process charts showing an example of a method for forming cell adhesion-inhibiting portions in the cell culture patterning substrate of the invention.

FIG. 7 is a schematic sectional view showing an example of the cell culture substrate of the invention.

FIGS. 8A and 8B are each a schematic sectional view for explaining a base material used in the cell culture patterning substrate of the invention.

### DESCRIPTION OF THE PREFERRED EMBODIMENT

The present invention relates to such as a cell culture patterning substrate on which cells can be adhered in highly precise pattern, a patterning substrate that is used to form the cell culture patterning substrate, a coating liquid for patterning substrate that is used to form the patterning substrate, and a cell culture substrate on which cells are adhered in highly precise pattern. Hereinafter, these will be explained below separately.

### A. Coating Liquid for Patterning Substrate

First, the coating liquid for patterning substrate of the present invention is described. This coating liquid for patterning substrate is a liquid comprising a cell adhesion-inhibiting material which has cell adhesion-inhibiting properties of inhibiting adhesion to cells and is decomposed or denatured by action of a photocatalyst on the basis of irradiation with energy, and a cell adhesive material which has cell adhesive properties.

The coating liquid for patterning substrate of the invention comprises the cell adhesion-inhibiting material, which has cell adhesion-inhibiting properties; therefore, in the case of applying the coating liquid for patterning substrate onto a layer containing a photocatalyst so as to form a layer, the layer can be rendered a layer low in cell adhesive properties. When this layer is irradiated with energy, the cell adhesion-inhibiting material is decomposed or denatured so as not to inhibit adhesion to cells; further, the layer also contains the cell adhesive material. For these reasons, the region irradiated with the energy can be rendered a region good in cell adhesive properties. Accordingly, a region good in cell adhesive properties and a region low in cell adhesive properties can easily be formed without using any complicated step or any treating solution or the like that produces a bad effect on cells.

The following will describe each of the constituents used in the above-mentioned coating liquid for patterning substrate.

### 1. Cell Adhesion-Inhibiting Material

A cell adhesion-inhibiting material that is used in the coating liquid for patterning substrate according to the present invention will be explained. The cell adhesion-inhibiting material used in the coating liquid for patterning substrate according to the invention, as far as it has cell adhesion-inhibiting properties that inhibit cells from adhering and can be decomposed or denatured by action of the photocatalyst on the basis of irradiation with energy, is not particularly restricted in the kind and the like.

Here, the wording “has the cell adhesion-inhibiting properties” means to have a property that inhibits cells from adhering to the cell adhesion-inhibiting material, and, when cell adhesive properties is different depending on the kind of cells and the like, means to have the property of inhibiting from adhering with a target cell.

As the cell adhesion-inhibiting material used in the present invention, one that has such cell adhesion-inhibiting properties and can be decomposed or denatured by action of the photocatalyst on the basis of irradiation with energy to lose the cell adhesion-inhibiting properties or to become good in the cell adhesive properties can be used.

As the cell adhesion-inhibiting material, materials high in, for instance, the hydration ability can be used. When the material high in the hydration ability is used, water molecules gather in the vicinity of the cell adhesion-inhibiting material to form a hydrated layer. Normally, such material high in the hydration ability is higher in adhesive properties with water molecules than that with the cell; accordingly, the cells cannot adhere to the material high in the hydration ability, resulting in one low in the cell adhesive properties. Here, “the hydration ability” means a property of hydrolyzing with water molecules, and “the hydration ability being high” means that it can readily hydrolyze with water molecules.

Examples of the material which is high in hydration ability and is used as the cell adhesion-inhibiting material include polyethylene glycol, amphoteric ion materials having a betaine structure, and phospholipid-containing materials such as poly(2-methacryloyloxyethyl)phosphorylcholine. When such a material is used as the cell adhesion-inhibiting material and is irradiated with energy, the cell adhesion-inhibiting material is decomposed, denatured, or the like by action of the photocatalyst so that the hydration layer comes off from the surface to render this material a material having no cell adhesion-inhibiting properties.

As the cell adhesion-inhibiting material, a material having constitution water on the surface thereof may be used. In the case of using such a material as the cell adhesion-inhibiting material, cells are inhibited from adhering onto this material by action of the constitution water so that adhesive properties between the material and the cells can be made low. Examples of the material which has constitution water and is used as the cell adhesion-inhibiting material in the invention include amphoteric ion materials having a betaine structure, and poly (2-methoxyethyl)acrylate. In the case of using such a material as the cell adhesion-inhibiting material and irradiating the material with energy, this material is decomposed, denatured, or the like by action of the photocatalyst so that the constitution water is removed from the surface to render the material a material not having cell adhesion-inhibiting properties.

A surfactant having a water repellent or oil repellent organic group that can be decomposed by action of the photocatalyst also can be used. As such surfactant, hydrocarbons base surfactants such as the respective series of NIKKO L, BL, BC, BO, and BB manufactured by Nikko Chemicals Co., Ltd., and fluorine base or silicone base nonionic surfactants such as ZONYL FSN and FSO manufacture by Du Pont Kabushiki Kaisya, Surflon S-141 and 145 manufactured by ASAHI GLASS CO., LTD., Megaface F-141 and 144 manufactured by DAINIPPON INK AND CHEMICALS, Inc., FTERGENT F-200 and F251 manufactured by NEOS, UNIDYNE DS-401 and 402 manufactured by DAIKIN INDUSTRIES, Ltd., and Fluorad FC-170 and 176 manufactured by 3M can be cited, and furthermore cationic surfactants, anionic surfactants and amphoteric surfactants also can be used.

At the time of applying the coating liquid for patterning substrate, using such a material as the cell adhesion-inhibiting material so as to form a layer, the cell adhesion-inhibiting material is oriented or unevenly distributed on the surface thereof. This makes it possible to make the water repellency or the oil repellency of the surface high so as to render the surface a surface having a small interaction with cells to exhibit low cell adhesive properties. In the case of irradiating this layer with energy, the cell adhesion-inhibiting material is easily decomposed by action of the photocatalyst so that the layer or the like that contains the photocatalyst is made exposed to produce a member not having cell adhesion-inhibiting properties.

In the present invention, as the cell adhesion-inhibiting material, one that can be good in the cell adhesive properties by action of the photocatalyst on the basis of irradiation with energy is particularly preferably used. As such cell adhesion-inhibiting materials, for instance, materials having the oil repellency or water repellency can be cited.

In the case of, as the cell adhesion-inhibiting material, a material having the water repellency or the oil repellency being used, owing to the water repellency or the oil repellency of the cell adhesion-inhibiting material, one in which an interaction such as a hydrophobic interaction between the cells and the cell adhesion-inhibiting material is small and the cell adhesive properties is low can be obtained.

As a material having such water repellency or the oil repellency, for instance, one where a skeleton has such a high bonding energy that cannot be decomposed by action of the photocatalyst and that has a water repellent or oil repellent organic substitution group that can be decomposed by action of the photocatalyst can be cited.

As one that has the skeleton having such high bonding energy that cannot be decomposed by action of the photocatalyst and the water repellent or oil repellent organic substitution group that can be decomposed by action of the photocatalyst, for instance, (1) organopolysiloxane that exhibits large strength by hydrolyzing or poly condensating chloro, alkoxysilane or the like owing to a sol-gel reaction and the like, and (2) organopolysiloxane in which reactive silicones are crosslinked can be cited.

In the case of the (1), it is preferable to be organopolysiloxane that is a hydrolysis condensate or cohydrolysis condensate of at least one kind of silicon compounds expressed by a general formula:

YₙSiX₍₄₋ₙ₎

(Here, Y denotes an alkyl group, fluoroalkyl group, vinyl group, amino group, phenyl group or epoxy group, or organic groups including these, and X denotes an alkoxyl group, acetyl group or halogen. n is an integer of 0 to 3). The number of carbons of the group expressed with Y is preferably in the range of 1 to 20, and the alkoxy group expressed with X is preferably a methoxy group, ethoxy group, propoxy group or butoxy group.

As the organic groups, in particular, polysiloxane containing a fluoroalkyl group can be preferably used. Specifically, a hydrolysis condensate or cohydrolysis condensate of at least one kind of fluoroalkylsilanes below can be cited. Ones generally known as the fluorinated silane coupling agents can be used.

### CF₃(CF₂)₃CH₂CH₂Si(OCH 3)₃;

### CF₃(CF₂)₅CH₂CH₂Si(OCH₃)₃;

### CF₃(CF₂)₇CH₂CH₂Si(OCH₃)₃;

### CF₃(CF₂)₉CH₂CH₂Si(OCH₃)₃;

### (CF₃)₂CF (CF₂)₄CH₂CH₂Si(OCH₃)₃;

### (CF₃)₂CF (CF₂)₆CH₂CH₂Si(OCH₃)₃;

### (CF₃)₂CF (CF₂)₈CH₂CH₂Si(OCH₃)₃;

### CF₃(C₆H₄)C₂H₄Si(OCH₃)₃;

### CF₃(CF₂) 3 (C₆H₄)C₂H₄Si(OCH₃)₃;

### CF₃(CF₂) 5 (C₆H₄)C₂H₄Si(OCH₃)₃;

### CF₃(CF₂) 7 (C₆H₄)C₂H₄Si(OCH₃)₃;

### CF₃(CF₂)₃CH₂CH₂SiCH₃(OCH₃)₂;

### CF₃(CF₂)₅CH₂CH₂SiCH₃(OCH₃)₂;

### CF₃(CF₂)₇CH₂CH₂SiCH₃(OCH₃)₂;

### CF₃(CF₂)₉CH₂CH₂SiCH₃(OCH₃)₂;

### (CF₃)₂CF(CF₂)₄CH₂CH₂SiCH₃(OCH₃)₂;

### (CF₃)₂CF(CF₂)₆CH₂CH₂Si CH₃(OCH₃)₂;

### (CF₃)₂CF(CF₂)₈CH₂CH₂Si CH₃(OCH₃)₂;

### CF₃(C₆H₄)C₂H₄SiCH₃(OCH₃)₂;

### CF₃(CF₂) 3 (C₆H₄)C₂H₄SiCH₃(OCH₃)₂;

### CF₃(CF₂) 5 (C₆H₄)C₂H₄SiCH₃(OCH₃)₂;

### CF₃(CF₂) 7 (C₆H₄)C₂H₄SiCH₃(OCH₃)₂;

### CF₃(CF₂)₃CH₂CH₂Si(OCH₂CH₃)₃;

### CF₃(CF₂)₅CH₂CH₂Si(OCH₂CH₃)₃;

### CF₃(CF₂)₇CH₂CH₂Si(OCH₂CH₃)₃;

### CF₃(CF₂)₉CH₂CH₂Si(OCH₂CH₃)₃;

### CF₃(CF₂)₇SO₂N(C₂H₅)C₂H₄CH₂Si(OCH₃)₃

When polysiloxane containing such a fluoroalkyl group mentioned above is used as the cell adhesion-inhibiting material, one high in the water repellency or oil repellency can be formed, resulting in rendering one small in the interaction with cells and low in the cell adhesive properties. Furthermore, when energy is irradiated on such a material, readily, fluorine and the like can be removed and an OH group and the like can be introduced on a surface to render the interaction with cells large; accordingly, the cell adhesive properties can be made good.

As the reactive silicone according to the (2), compounds having a skeleton expressed by a general formula below can be cited.

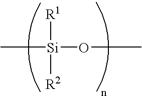

In the above general formula, n denotes an integer of 2 or more, R¹ and R² each represents a substituted or nonsubstituted alkyl group, alkenyl group, aryl group or cyanoalkyl group having 1 to 18 carbons, and a vinyl, phenyl and halogenated phenyl occupy 40% or less by mole ratio to a total mole. Furthermore, one in which R¹ and R² each is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain.

Together with the organopolysiloxane, a stable organosilicon compound that does not cause a crosslinking reaction such as dimethylpolysiloxane may be blended separately.

By use of a reactive silicone as described above, the cell adhesion-inhibiting material can be rendered a material which has a high water repellency or oil repellency and gives a small interaction with cells to exhibit low cell adhesive properties. In the case of irradiating a material as described above with energy, substituents thereof are easily removed and OH groups can be introduced onto the surface. Therefore, the surface free energy can be controlled. As a result, the interaction of the surface with cells can be made large to make the cell adhesive properties thereof good.

In the case of using the material having water repellency or oil repellency as the cell adhesion-inhibiting material, the material is usually a material the contact angle of which with water is preferably 80° or more, more preferably from 100 to 130°. According to this, the cell adhesive properties thereof can be made low. The upper limit of the angle is the upper limit of the contact angle of the cell adhesion-inhibiting material with water on a flat base material. In the case of measuring the contact angle of the cell adhesion-inhibiting material with water on, for example, a base material having irregularities, the upper limit may be about 160° as described in, for example, Ogawa et al., Japanese Journal of Applied Physics, vols. 2 and 3, L614-L615 (1993).

When the cell adhesion-inhibiting material is irradiated with energy so as to be rendered a material having cell adhesive properties, it is preferred to irradiate the energy to set the contact angle with water into the range of 10 to 40°, in particular 15 to 30° C. This makes it possible to render the cell adhesion-inhibiting material a material high in cell adhesive properties.

The contact angle with water referred to herein is a result obtained by using a contact angle measuring device (CA-Z model, manufactured by Kyowa Interface Science Co., Ltd.) to measure the contact angle of the material with water or a liquid having a contact angle equivalent to that of water (after 30 seconds from the time when droplets of the liquid are dropped down from its micro syringe), or a value obtained from a graph prepared from the result.

The amount of the above-mentioned cell adhesion-inhibiting material is appropriately optimized in accordance with the cell adhesion-inhibiting ability of the material. Usually, the content by percentage thereof in the coating liquid for patterning substrate is preferably from 0.01 to 30% by weight, more preferably from 0.1 to 10% by weight. This makes it possible to render the region comprising the cell adhesion-inhibiting material a region low in cell adhesive properties.

When energy is irradiated on the cell adhesion-inhibiting material such as mentioned above to decompose or denature and thereby to use as one good in the cell adhesive properties, the energy may be irradiated to decompose or denature the cell adhesion-inhibiting material to an extent that makes the adhesive properties with target cell good, and there is no need of completely decomposing or denaturing the cell adhesion-inhibiting material.

### 2. Cell Adhesive Material

The following will describe the cell adhesive material used in the coating liquid for patterning substrate of the invention. This cell adhesive material is not limited to any special kind if the material has cell adhesive properties at least after the material is irradiated with energy. The cell adhesive material may be, for example, a material which comes to have good cell adhesive properties before the material is irradiated with energy, or a material having good cell adhesive properties by action of a photocatalyst on the basis of irradiation with energy. The wording “having cell adhesive properties” means “adhereing” satisfactorily onto cells. When a material has different cell adhesive properties in accordance with the kinds of cells, the wording means “adhereing satisfactorily onto target one(s) out of the cells”.

When the cell adhesive material has good cell adhesive properties at least after the material is irradiated with energy in the invention, the adhesion thereof to cells may be made good by noncovalent bonding, such as hydrophobic interaction, electrostatic interaction, hydrogen bonding or van der Waals' force, or by biological interaction such as biotin-avidin or antigen-antibody interaction.

As a material that has the cell adhesive properties owing to the noncovalent bonding, specifically, basic polymers such as polystyrene, hydrophilic polystyrene and poly (N-isopropylacrylamide), polylysine, basic compounds such as aminopropyltriethoxysilane, N-(2-aminoethyl)-3-aminopropyltrimethoxysilane, condensates, and the like including these can be cited.

As a material having the cell adhesive properties owing to the biological characteristics, specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and the like can be cited.

In the invention, the amount of the cell adhesive material is appropriately optimized in accordance with the cell adhering ability of the material. Usually, the content by percentage thereof in the coating liquid for patterning substrate is preferably from 0.0001 to 30% by weight, more preferably from 0.001 to 10% by weight. This makes it possible that at the time of applying the coating liquid for patterning substrate to form a layer, the cell adhesive properties of the region irradiated with energy are made better. In the case of using, as the cell adhesive material, the material having good cell adhesive properties before the irradiation thereof with energy, it is preferred that the material is contained to such a degree that the cell adhesion-inhibiting properties of the cell adhesion-inhibiting material are not inhibited in the region not irradiated with energy after the coating liquid for patterning substrate is applied to form a layer.

### 3. Coating Liquid for Patterning Substrate

The following will describe the coating liquid for patterning substrate of the present invention. This coating liquid for patterning substrate is not limited to any special kind if the coating liquid contains the above-mentioned cell adhesion-inhibiting material and cell adhesive material. If necessary, the coating liquid may appropriately contain a binder and other components. The incorporation of the binder makes it possible to make easy the application of the coating liquid for patterning substrate onto, for example, a layer or the like which contains a photocatalyst and further give various properties, such as strength and resistance, to the formed layer. The binder may be appropriately selected in accordance with the use purpose of the coating liquid for patterning substrate. In the invention, the cell adhesive material or cell adhesion-inhibiting material may fulfill the role of this binder.

### B. Patterning Substrate

The following will describe the patterning substrate of the present invention. This patterning substrate comprises a base material, a photocatalyst-containing layer which is formed on the base material and comprises at least a photocatalyst, and a cell adhesion-inhibiting layer which is formed on the photocatalyst-containing layer and comprises a cell adhesion-inhibiting material that has cell adhesion-inhibiting properties of inhibiting adhesion to cells and is decomposed or denatured by action of the photocatalyst on the basis of irradiation with energy.

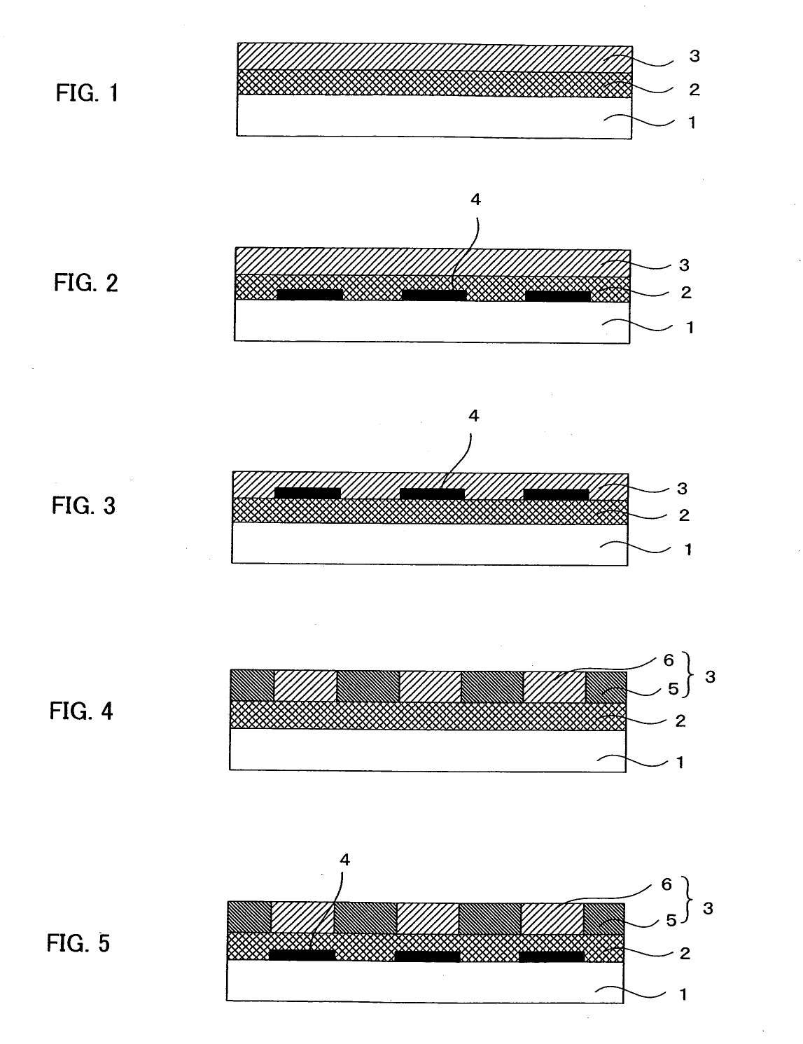

As shown in, for example, FIG. 1, the patterning substrate of the present invention is a substrate having a base material 1, a photocatalyst-containing layer 2 formed on the base material 1, and a cell adhesion-inhibiting layer 3 formed on the photocatalyst-containing layer 2.

According to the invention, cells are not permitted to adhere onto the cell adhesion-inhibiting layer since this layer comprises the cell adhesion-inhibiting material having cell adhesion-inhibiting properties. In the case of irradiating the cell adhesion-inhibiting layer with energy, the cell adhesion-inhibiting material is decomposed or denatured by action of the photocatalyst in the photocatalyst-containing layer on the basis of irradiation with energy so that cells can adhere. Accordingly, the irradiation of energy into a pattern form, makes it possible to form easily a region good in cell adhesive properties, where the cell adhesion-inhibiting material is decomposed and removed, and a region low in cell adhesive properties, where the cell adhesion-inhibiting material remains, without requiring any special device or complicated step.

As shown in, for example, FIG. 2, light-shielding portions 4 may be formed on the base material 1 in the patterning substrate of the invention. As shown in, for example, FIG. 3, light-shielding portions 4 may be formed on the photocatalyst-containing layer 2. The formation of such light-shielding portions makes it possible to render the photocatalyst inside regions where the light-shielding portions are formed a photocatalyst which is not excited and further render the cell adhesion-inhibiting material contained in the cell adhesion-inhibiting layer on the same regions a material which is not decomposed or removed when the energy is irradiated onto the patterning substrate from its base material. It is therefore possible to form regions good in cell adhesive properties and regions poor in cell adhesive properties without using a photomask or the like.

The following will describe each of the constituents of the patterning substrate of the present invention.

### 1. Cell Adhesion-Inhibiting Layer

The cell adhesion-inhibiting layer used in the patterning substrate of the invention is first described. This cell adhesion-inhibiting layer is not limited to any special kind if the layer is a layer having cell adhesion-inhibiting properties of inhibiting adhesion to cells and comprising a cell adhesion-inhibiting material which is decomposed or denatured by action of a photocatalyst on the basis of irradiation with energy.

This cell adhesion-inhibiting layer can be formed by applying a coating liquid or the like that comprises the above-mentioned cell adhesion-inhibiting material into the form of a layer. It is particularly preferred in the invention that the layer is a layer formed by applying a coating liquid for patterning substrate as described in the item “A. Coating Liquid for Patterning Substrate”, which comprises the cell adhesion-inhibiting material and a cell adhesive material as described above. This makes it possible that at the time of irradiating the cell adhesion-inhibiting layer with energy, cell adhesive properties of the region irradiated with the energy are made better.

The method for forming such a cell adhesion-inhibiting layer may be a method of applying the above-mentioned coating liquid for patterning substrate in an ordinary coating manner or the like. Specifically, the coating manner which can be used may be spin coating, spray coating, dip coating, roll coating, bead coating or the like. An adsorbing process also can be preferably used.

When concave portions are formed in the base material which will be described later as one of the constituents, it is possible to use: a casting process of forming the photocatalyst-containing layer which will be described later as one of the constituents in the concave portions of the base material, dropping down the coating liquid for patterning substrate or the like into the concave portions, and drying the liquid so as to form the cell adhesion-inhibiting layer; an adsorbing process of forming a photocatalyst-containing layer, which will be described later, in the concave portions of the base material, dropping down the coating liquid for patterning substrate or the like into the concave portions, and washing the surface after a predetermined time; or some other process.

The cell adhesion-inhibiting material, the cell adhesive material and so on which are used in the cell adhesion-inhibiting layer used in the invention are the same as described in the above-mentioned item “A. Coating Liquid for Patterning Substrate”. Thus, the description thereof is omitted herein.

The film thickness of the cell adhesion-inhibiting layer is appropriately selected in accordance with the kind of the patterning substrate and others, and is usually from about 0.001 to 1 μm, preferably from about 0.005 to 0.1 μm.

### 2. Photocatalyst-Containing Layer

The following will describe the photocatalyst-containing layer used in the patterning substrate of the invention. This photocatalyst-containing layer is not limited to any special kind if the layer is a layer comprising at least a photocatalyst, and may be a layer made only of a photocatalyst, or a layer which also comprises other components such as a binder.

The action mechanism of the photocatalyst, a typical example thereof being titanium oxide, which will be described later, in this photocatalyst-containing layer is not necessarily clear, but it has been considered as follows: carriers generated by the irradiation thereof with light react directly with a compound in the vicinity thereof, or are combined with active oxygen species generated in the presence of oxygen or water, thereby changing the chemical structure of organic material. In the present invention, the carriers would act onto the cell adhesion-inhibiting material in the cell adhesion-inhibiting layer formed on the photocatalyst-containing layer.

As the photocatalyst that can be used in the present invention, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) andiron oxide (Fe₂O₃) that are known as photo-semiconductors can be cited. These can be used singularly or in combination of at least two kinds.

In the present invention, in particular, titanium dioxide, owing to a large band gap, chemical stability, non-toxicity, and easy availability, can be preferably used. There are two types of titanium dioxide, anatase type and rutile type, and both can be used in the invention; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by Ishihara Sangyo Kaisha, Ltd., average particle diameter: 7 nm, and trade name: ST-KO1, manufactured by Ishihara Sangyo Kaisha, Ltd.), an anatase titania sol of nitric acid deflocculation type (trade name: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. An average particle diameter of the photocatalyst is preferably 50 nm or less, and one having an average particle diameter of 20 nm or less can be particularly preferably used.

It is advantageous from the viewpoint of costs to use the photocatalyst-containing layer made only of a photocatalyst since the efficiency of decomposing or denaturing the cell adhesion-inhibiting material in the cell adhesion-inhibiting layer is improved to make the time for the treatment shorter. On the other hand, the use of the photocatalyst-containing layer made of a photocatalyst and a binder gives an advantage of making the formation of the photocatalyst-containing layer easy.

An example of the method for forming the photocatalyst-containing layer made only of a photocatalyst may be a vacuum film-forming method such as sputtering, CVD or vacuum vapor deposition. The formation of the photocatalyst-containing layer by the vacuum film-forming method makes it possible to render the layer a homogeneous photocatalyst-containing layer made only of a photocatalyst, thereby decomposing or denaturing the cell adhesion-inhibiting material homogeneously, and further thereby making the decomposition or denaturation of the cell adhesion-inhibiting material more effective in this case than in the case of using a binder also since the layer is made only of the photocatalyst. When concave portions are formed on the base material which will be described later as well, the photocatalyst-containing layer can be formed by the above-mentioned method.

Another example of the method for forming the photocatalyst-containing layer made only of a photocatalyst, is the following method: in the case that the photocatalyst is, for example, titanium dioxide, amorphous titania is formed on the base material and next fired so as to phase-change the titania to crystalline titania. The amorphous titania used in this case can be obtained, for example, by hydrolysis or dehydration condensation of an inorganic salt of titanium, such as titanium tetrachloride or titanium sulfate, or hydrolysis or dehydration condensation of an organic titanium compound, such as tetraethoxytitanium, tetraisopropoxytitanium, tetra-n-propoxytitanium, tetrabutoxytitanium or tetramethoxytitanium, in the presence of an acid. Next, the resultant is fired at 400 to 500° C. so as to be denatured to anatase type titania, and fired at 600 to 700° C. so as to be denatured to rutile type titania.

In the case of using a binder together with the above-mentioned photocatalyst, the binder is preferably a binder having a high bonding energy, wherein its main skeleton is not decomposed by photoexcitation of the photocatalyst. Examples of such a binder include the organopolysiloxanes described in the above-mentioned item “Cell Adhesion-Inhibiting Layer”.

In the case of using such an organopolysiloxane as the binder, the photocatalyst-containing layer can be formed by dispersing a photocatalyst, the organopolysiloxane as the binder, and optional additives if needed into a solvent to prepare a coating liquid, and applying this coating liquid onto the base material which will be described later. The used solvent is preferably an alcoholic based organic solvent such as ethanol or isopropanol. The application can be performed by a known coating method such as spin coating, spray coating, dip coating, roll coating, or bead coating. When the coating liquid contains an ultraviolet curable component as the binder, the photocatalyst-containing layer can be formed by curing the coating liquid through the irradiation of ultraviolet rays onto the liquid.

When concave portions are formed on the base material which will be described later, it is allowable to perform such as a casting method of dropping down the above-mentioned coating liquid or the like into the concave portions and then drying the liquid to form the photocatalyst-containing layer.

As the binder, an amorphous silica precursor can be used. This amorphous silica precursor is preferably a silicon compound represented by the general formula SiX₄, wherein X are a halogen, a methoxy group, an ethoxy group, an acetyl group or the like; a silanol which is a hydrolyzate thereof; or a polysiloxane having an average molecular weight of 3000 or less.

Specific examples thereof include such as tetraethoxysilane, tetraisopropoxysilane, tetra-n-propoxysilane, tetrabutoxysilane, and tetramethoxysilane. In this case, the photocatalyst-containing layer can be formed by dispersing the amorphous silica precursor and particles of a photocatalyst homogeneously into a non-aqueous solvent, hydrolyzing the precursor with water content in the air to form a silanol onto a transparent base material, and then subjecting the silanol to dehydration polycondensation at room temperature. When the dehydration polycondensation of the silanol is performed at 100° C. or higher, the polymerization degree of the silanol increases so that the strength of the film surface can be improved. A single kind or two or more kinds of this binding agent may be used.

The content by percentage of the photocatalyst in the photocatalyst-containing layer is from 5 to 60% by weight, preferably from 20 to 40% by weight. The thickness of the photocatalyst-containing layer is preferably from 0.05 to 10 μm.

Besides the above-mentioned photocatalyst and binder, the surfactant and so on described in the above-mentioned item “Cell Adhesion-Inhibiting Layer” can be incorporated into the photocatalyst-containing layer.

It is preferred in the invention that the photocatalyst-containing layer has a surface high in cell adhesive properties. According to this, when the cell adhesion-inhibiting layer is decomposed or the like to make the photocatalyst-containing layer exposed, the region can be rendered a region high in cell adhesive properties.

In the invention, a light-shielding portion may be formed on the photocatalyst-containing layer as described above. This makes it possible that at the time of irradiating the entire surface of the cell adhesion-inhibiting layer with energy, the photocatalyst inside the region where the light-shielding portion is formed is not excited so that the cell adhesion-inhibiting material contained in the cell adhesion-inhibiting layer outside the region where the light-shielding portion is formed is decomposed or denatured. In this case, the direction in which the energy is irradiated is not particularly limited since the photocatalyst in the same region where the light-shielding portion is formed is not excited.

Such light-shielding portions are not limited to any special kind if the portions make it possible to shield energy irradiated on the patterning substrate. The light-shielding portions may be formed, for example, by forming a thin film made of a metal such as chromium to have a thickness of about 1000 to 2000 Å by sputtering, vacuum vapor deposition or the like, and then patterning this thin film. The method for the patterning may be an ordinary patterning method such as sputtering.

The patterning method may be a method of forming, into a pattern form, a layer wherein shielding particles of carbon fine particles, a metal oxide, an inorganic pigment, an organic pigment or the like are incorporated into a resin binder. Examples of the used resin binder include a resin such as polyimide resin, acrylic resin, epoxy resin, polyacrylamide, polyvinyl alcohol, gelatin, casein or cellulose; mixtures made of two ore more thereof; photosensitive resins; and O/W emulsion type resin compositions, such as a material obtained by emulsifying a reactive silicone. The thickness of the light-shielding portions made of such a resin may be from 0.5 to 10 μm. The method for patterning the resin light-shielding portions may be an ordinarily-used method such as photolithography or printing.

### 3. Base Material

The following will describe the base material used in the present invention. This base material is not limited to any special kind if the material is a material on which the photocatalyst-containing layer can be formed. For example, the base material may be made of an inorganic material such as metal, glass or silicon, or an organic material, a typical example of which is plastic.

The flexibility and the like of the base material are properly selected according to the kind of the patterning substrate, applications, or the like. Furthermore, the transparency of the base material is properly selected depending on such as the kind of the patterning substrate, or a direction in which energy that is irradiated to decompose or denature the cell adhesion-inhibiting material is irradiated. For instance, when the base material has such as the light-shielding portion and the energy is irradiated from a base material side and the like, the base material has the transparency.

In the invention, the base material may be a flat base material or a base material wherein one or more concave portions are formed. The base material may be a base material wherein a single concave portion is formed as shown in FIG. 8A, or a base material wherein plural concave portions are formed as shown in FIG. 8B.

At this time, side walls of the base material having the concave portion(s) may be treated in such a manner that the photocatalyst-containing layer or cell adhesion-inhibiting layer will not be formed thereon. Examples of the method for such a treatment include a method of using a mask or the like to cause a material having liquid repellency to adhere only onto the side walls by CVD; and a method of causing a material having liquid repellency to adhere onto the entire surface of the concave portion(s) and then using a cylindrical mask or the like to conduct ultraviolet ray treatment, plasma treatment or some other treatment, thereby making the bottom face(s) of the concave portion(s) lyophilic.

In the invention, the base material may be washed with a medical liquid, such as an alkali solution, and may be subjected to dry washing, such as oxygen plasma treatment or ultraviolet treatment. In this case, the wettability of the coating liquid or the like for forming the photocatalyst-containing layer is improved. Furthermore, this case has an advantage that the adhesive property of the base material to the photocatalyst-containing layer is improved since reactive functional groups are arranged on the surface of the base material.

As described above, one or more light-shielding portions may be formed on the base material of the invention. The light-shielding portions formed on the base material are equivalent to those described in the above-mentioned item “Photocatalyst-Containing Layer”. Thus, the description thereof is omitted herein. The light-shielding portions may be ones formed on the surface of the side at which the photocatalyst-containing layer is formed or ones formed on the opposite surface.

In the case of forming the light-shielding portions on the base material and forming the photocatalyst-containing layer on the light-shielding portions, a primer layer may be formed between the photocatalyst-containing layer and the light-shielding portions. The effect and function of this primer layer are not necessarily clear, but would be as follows: the primer layer exhibits a function of preventing the diffusion of impurities from openings which are present in the light-shielding portions and between the light-shielding portions, the impurities being factors for blocking the decomposition or denaturation of the cell adhesion-inhibiting material in the cell adhesion-inhibiting layer by action of the photocatalyst, in particular, residues generated when the light-shielding portions are patterned, or metal, metal ion impurities, or the like. Accordingly, the formation of the primer layer makes it possible to decompose or denature the cell adhesion-inhibiting material with high sensitivity so that a high resolution pattern can be obtained.

The primer layer in the present invention inhibits the impurities present not only in the light-shielding portion but also in the openings formed between the light-shielding portions from adversely affecting on an action of the photocatalyst; accordingly, the primer layer is preferably formed over an entire surface of the light-shielding portion including the openings.

The primer layer in the invention, as far as it is formed so that the light-shielding portion and the photocatalyst-containing layer may not be brought into contact, is not particularly restricted.

A material that forms the primer layer, though not particularly restricted, is preferably an inorganic material that is not likely to be decomposed by action of the photocatalyst. Specifically, amorphous silica can be cited. When such amorphous silica is used, a precursor of the amorphous silica is preferably a silicon compound that is represented by a general formula, SiX₄, X being halogen, methoxy group, ethoxy group, acetyl group or the like, silanol that is a hydrolysate thereof, or polysiloxane having an average molecular weight of 3000 or less.

A film thickness of the primer layer is preferably in the range of 0.001 to 1 μm and particularly preferably in the range of 0.001 to 0.1 μm.

### 4. Patterning Substrate

Subsequently, a patterning substrate in the invention will be explained. The patterning substrate in the invention, as far as it is one in which the above-mentioned photocatalyst-containing layer is formed on the base material and further the cell adhesion-inhibiting layer is formed on the photocatalyst-containing layer, is not particularly restricted. For instance, as needs arise, one in which another layer is further laminated and the like can be used.

### C. Cell Culture Patterning Substrate

The following will describe the cell culture patterning substrate of the present invention. This cell culture patterning substrate is a substrate wherein the cell adhesion-inhibiting layer of the above-mentioned patterning substrate comprises a cell adhesion portion where the cell adhesion-inhibiting material is decomposed or denatured in a pattern form, and a cell adhesion-inhibiting portion which is a region other than the cell adhesion portion.

As shown in for example, FIG. 4, an example of the cell culture patterning substrate of the invention has a base material 1, a photocatalyst-containing layer 2 formed on the base material 1, and a cell adhesion-inhibiting layer 3 formed on the photocatalyst-containing layer 2, wherein the cell adhesion-inhibiting layer 3 has cell adhesion portions 5 and cell adhesion-inhibiting portions 6.

According to the invention, in the cell adhesion portions, the cell adhesion-inhibiting material is decomposed or denatured; therefore, the cell adhesive properties thereof are not inhibited so that the portions can be rendered portions high in cell adhesive properties. The cell adhesion-inhibiting portions comprise a cell adhesion-inhibiting material to exhibit low cell adhesive properties. It is therefore possible to cause cells to adhere only onto the cell adhesion portions without using any complicated step, or any treating solution or the like that produces a bad effect on the cells.

The cell adhesion-inhibiting portions are regions comprising a cell adhesion-inhibiting material which has cell adhesion-inhibiting properties of inhibiting adhesion to cells and is decomposed or denatured by action of a photocatalyst on the basis of irradiation with energy, and are regions low in adhesive properties to target cells.

On the other hand, the cell adhesion portions are regions having cell adhesive properties where the cell adhesion-inhibiting material is decomposed or denatured. The wording “the cell adhesion-inhibiting material is decomposed or denatured” means that the cell adhesion-inhibiting material is not contained or the cell adhesion-inhibiting material is contained in a smaller amount than the amount of the cell adhesion-inhibiting material contained in the cell adhesion-inhibiting portion. When the cell adhesion-inhibiting material is, for example, a material which can be decomposed by action of a photocatalyst on the basis of irradiation with energy, the wording means that a small amount of the cell adhesion-inhibiting material is contained in the cell adhesion portions; a decomposition product or the like of the cell adhesion-inhibiting material is contained; the cell adhesion-inhibiting material is completely decomposed and removed so that the photocatalyst-containing layer is made exposed; or the like. When the cell adhesion-inhibiting material is a material which can be denatured by action of a photocatalyst on the basis of irradiation with energy, the wording means that a denatured product thereof or the like is contained in the cell adhesion portions. In the invention, it is preferred that the cell adhesion portions contain a cell adhesive material having cell adhesive properties at least after the material is irradiated with energy. This makes it possible to make higher the cell adhesive properties of the cell adhesion portions so as to cause the cells to adhere highly precisely only onto the cell adhesion portions.

In the invention, light-shielding portions 4 may be formed, on the base material 1, in the same pattern form as the cell adhesion-inhibiting portions 6 of the cell adhesion-inhibiting layer 3 have, as shown in, for example, FIG. 5. Similarly, light-shielding portions may be formed on the photocatalyst-containing layer. When such light-shielding portions are formed, the cell adhesion-inhibiting material only on regions where no light-shielding portions are formed can be decomposed or denatured, for example, by forming a patterning substrate as described in the above-mentioned item “B. Patterning Substrate” and then irradiating energy onto the entire surface thereof from the side of the substrate or the like. As a result, the above-mentioned cell adhesion portions can easily be formed.

The base material, the photocatalyst-containing layer and the cell adhesion-inhibiting layer used in the cell culture patterning substrate of the invention are equivalent to those described in the above-mentioned item “Patterning Substrate”. Thus, the description thereof is omitted herein. The following will describe the method for forming the cell adhesion portions.

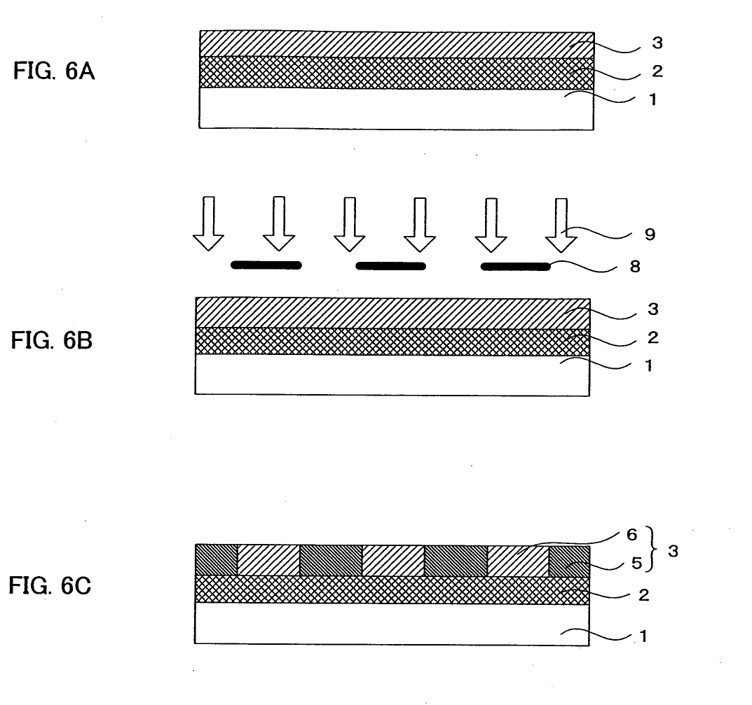

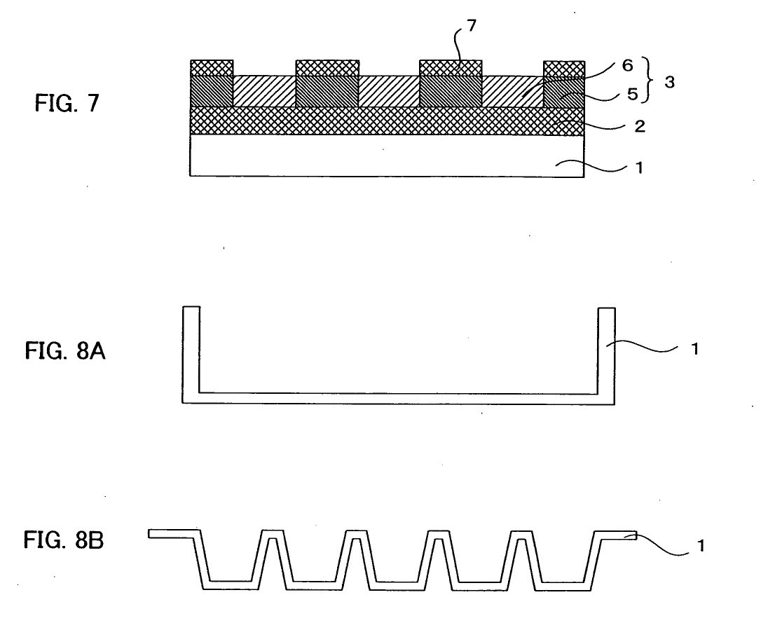

As shown in, for example, FIGS. 6A to 6C, a photocatalyst-containing layer 2 comprising at least a photocatalyst is formed on a base material 1, and then a coating liquid for patterning substrate as described in the above-mentioned item “A. Coating Liquid for Patterning Substrate” or the like is used to form a cell adhesion-inhibiting layer 3 comprising a cell adhesion-inhibiting material (FIG. 6A). Next, for example, a photomask 8 or the like is used to irradiate energy 9, into the form of a pattern for forming cell adhesion portions, onto this cell adhesion-inhibiting layer 3 (FIG. 6B). According to this, the cell adhesion-inhibiting material in the cell adhesion-inhibiting layer 3 inside the regions irradiated with the energy is decomposed or denatured by action of the photocatalyst contained in the photocatalyst-containing layer 2, so that the regions irradiated with the energy and the regions not irradiated with the energy can be rendered cell adhesion portions 5 and cell adhesion-inhibiting portions 6, respectively (FIG. 6C). The methods for forming the cell adhesion-inhibiting layer and the photocatalyst-containing layer, and others are equivalent to those described in the above-mentioned item “B. Patterning Substrate”. Thus, description thereof is omitted herein. As described above, it is preferred that the cell adhesion-inhibiting layer contains therein a cell adhesive material having cell adhesive properties at least after the material is irradiated with energy. This makes it possible to make the cell adhesive properties of the cell adhesion portions higher.

The energy irradiation (exposure) mentioned in the present invention is a concept that includes all energy line irradiation that can decompose or denature the cell adhesion-inhibiting material by action of a photocatalyst on the basis of irradiation with energy, and is not restricted to light irradiation.

Normally, a wavelength of light used in such energy irradiation is set in the range of 400 nm or less, and preferably in the range of 380 nm or less. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

The method for the exposure may be a method of using a laser, such as an excimer laser or YAG laser, to draw and radiate energy in a pattern form, besides a method of using a light source as described above to draw and radiate energy in a pattern form through a photomask. When the base material has light-shielding portions in the same pattern form as the cell adhesion portions have, as described above, the exposure can be performed by irradiating the entire surface of the cell culture patterning substrate from the base material side thereof. When the photocatalyst-containing layer has thereon light-shielding portions in the same pattern form as the cell adhesion-inhibiting portions have, the exposure can be performed by irradiating the entire surface from any direction. These cases have an advantage that a photomask, and positioning and other steps are unnecessary.

When the base material has one or more concave portions and the photocatalyst-containing layer and the cell adhesion-inhibiting layer are formed in the concave portion(s), as described above, the exposure may be performed by any one of the above-mentioned methods. For instance, in the case of the plural concave portions, for example, the exposure may be performed into the form of patterns different from each other for the individual concave portions. Examples of the method for performing the exposure into the form of patterns different from each other for the individual concave portions as described above include a method of arranging different masks for the individual concave portions to radiate energy; and a method of arranging a chromium mask, a stencil mask or the like at the tip of an optical fiber to radiate energy.

In order not to radiate any energy onto side walls of the concave portions, the method for the exposure may be, for example, a method of using a cylindrical mask to radiate energy only onto the bottom faces of the concave portions.

The irradiation quantity of the energy at the time of the energy-irradiation is set to a value necessary for decomposing or denaturing the cell adhesion-inhibiting material by action of the photocatalyst.

In this case, it is preferred to irradiate the photocatalyst-containing layer with the energy while heating the layer since the sensitivity can be raised so as to decompose or denature the cell adhesion-inhibiting material effectively. Specifically, the layer is preferably heated at a temperature of 30 to 80° C.

The energy irradiation that is carried out through a photomask in the invention, when the above-mentioned base material is transparent, may be carried out from either direction of a base material side or a cell adhesion-inhibiting layer side. On the other hand, when the base material is opaque, it is necessary to apply energy irradiation from a cell adhesion-inhibiting layer side.

In the invention, it is allowable to use, as the cell culture patterning substrate, such as a substrate obtained by forming the cell culture patterning substrate as described above, cutting one portion from the patterning substrate, and attaching this portion onto the bottom of a base material in a concave form, or the like.

### D. Cell Culture Substrate

Next, a cell culture substrate in the invention will be explained. A cell culture substrate in the invention is one in which cells are adhered onto the cell adhesion portion in the above-mentioned cell culture patterning substrate.

In the cell culture substrate in the invention, as shown in, for example, FIG. 7, cells 7 are adhered only onto a cell adhesion portion 5 of the cell adhesion-inhibiting layer 3, and, on a cell adhesion-inhibiting portion 6, cells 7 are not adhered.

In the invention, on the cell culture patterning substrate, a cell adhesion portion good in the cell adhesive properties and a cell adhesion-inhibiting portion that does not have the cell adhesive properties are formed. Accordingly, for instance, even when cells are coated over an entire surface of the cell culture patterning substrate, the cells can be adhered to the cell adhesion portion only and the cells on the cell adhesion-inhibiting portion can be readily removed. Thereby, without using any complicated step, any treating solution that produces a bad effect on the cells, or the like, the cell culture substrate can be readily formed.

Hereinafter, cells that are used in a cell culture substrate in the invention will be explained. Since an explanation of a cell culture patterning substrate is same as that in the “C. Cell Culture Patterning Substrate”, here it is omitted.

### (Cells)

As cells used in a cell culture substrate in the invention, as far as cells can adhere onto a cell adhesion portion of the cell culture patterning substrate but do not adhere to a cell adhesion-inhibiting portion, there is no particular restriction.

As cells used in the present invention, except for, for instance, non-adhesive cells such as nervous tissue, liver, kidney, pancreas, blood vessel, brain, cartilage and blood corpuscle, all tissues present in an organism and cells derived therefrom can be used. Furthermore, since even for generally non-adhesive cells, recently, in order to adhere and fix, a technology of modifying a cell membrane is devised; accordingly, as needs arise, the non-adhesive cells, when this technology is applied, can be used in the present invention.

The respective tissues such as mentioned above are formed of cells having various functions; accordingly, it is necessary to select desired cells to use. For instance, in the case of the liver, it is formed of, other than hepatocytes, epithelial cells, endothelial cells, Kupffer's cells, fibroblasts, and fat-storing cells and the like. In this case, since the cell adhesion-inhibiting properties with a cell adhesion-inhibiting material is different depending on the kinds of the cells, in accordance with a cell strain, a cell adhesion-inhibiting material used in the cell adhesion-inhibiting portion and a composition ratio thereof have to be selected.

A method of adhering cells to the cell adhesion portion, as far as it can adhere cells only on the cell adhesion portion of the cell culture patterning substrate that has the cell adhesion portion and the cell adhesion-inhibiting portion, is not particularly restricted. For instance, cells may be adhered by use of an ink jet printer, a manipulator or the like; however, a method in which after a cell suspension is disseminated to adhere cells on the cell adhesion portion, unnecessary cells on a cell adhesion-inhibiting portion are washed with a phosphate buffer to remove the cells is generally used. As such a method, a method described in, for instance, a reference literature, Kevin E. Healy et al., “Spatial distribution of mammalian cells dicated by material surface chemistry”, Biotech. Bioeng. (1994), p. 792 can be used.

The present invention is not limited to the above-mentioned embodiments. The embodiments are mere examples. All modifications which have substantially the same structure as the technical conception described in the claims of the invention and produce effects and advantages similar to those of the technical conception are included in the technical scope of the invention.

### EXAMPLES

Hereinafter, examples and comparative examples are shown and thereby the present invention will be more specifically described.

### Example 1

### Formation of a Photocatalyst-Containing Layer

Three grams of isopropyl alcohol, 0.4 g of an organosilane, TSL 8114 (manufactured by GE Toshiba Silicones), and 1.5 g of a photocatalyst inorganic coating agent, ST-K01 (manufactured by ISHIHARA SANGYO KAISYA, LTD.) were mixed, and then the mixture was heated at 100° C. for 20 minutes while stirred.

This solution was applied onto a glass substrate subjected to alkali treatment in advance by spin coating method, and the substrate was dried at 150° C. for 10 minutes to advance hydrolysis and polycondensation reaction, thereby forming, on the substrate, a photocatalyst-containing layer, 0.2 μm in thickness, wherein the photocatalyst was strongly fixed into an organopolysiloxane.

### (Formation of a Cell Adhesion-Inhibiting Layer)

A solution of 5 g of isopropyl alcohol, 0.4 g of an organosilane, TSL8114 (manufactured by GE Toshiba Silicones), and 0.04 g of a fluoroalkylsilane, TSL8233 (manufactured by GE Toshiba Silicones) was applied onto this substrate by spin coating. Thereafter, the substrate was dried at 150° C. for 10 minutes to form a cell adhesion-inhibiting layer.

### (Patterning of the Patterning Substrate)

This substrate was irradiated through a photomask with ultraviolet rays at 6 J/cm² (wavelength: 254 nm) from a mercury lamp, so as to yield a cell culture patterning substrate having a surface patterned to have unexposed portions having cell adhesion-inhibiting properties and have exposed portions having cell adhesive properties.

### (Cell Adhesion Process)

About the process of experiments for culturing cells originating from various kinds of tissues, details thereof are described in, for example, “Soshikibaiyo no Gijyutsu, Dai San Han, Kiso”, edited by The Japanese Tissue Culture Association and published by Asakura Shoten, and other documents. In the present application, rat hepatocytes were used to evaluate the substrate.

A liver extracted from a rat was transferred into a Petri dish, and the liver was cut into pieces 5 mm in size with a scalpel. Thereto was added 20 ml of a DMEM culture medium, and the pieces were lightly suspended with a pipette. Thereafter, the suspension was filtrated with a cell filter. The resultant cell-coarsely-dispersed suspension was subjected to centrifugation at 500 to 600 rpm for 90 seconds, and the supernatant was sucked to be removed. A new DMEM culture medium was added to the remaining cells, and the resultant was again subjected to centrifugation. This operation was repeated three times to yield substantially homogenous hepatocytes. To the resultant hepatocytes was added 20 ml of a DMEM culture medium, and the cells were suspended therein to prepare a hepatocytes suspension.

Next, 900 ml of distilled water was added to 14.12 g of a Waymouth MB 752/1 culture medium (containing L-glutamine but containing no NaHCO₃) (manufactured by GIBCO). To this were added 2.24 g of NaHCO₃, 10 ml of an amphotericin B solution (ICN), and 10 ml of a penicillin streptomycin solution (manufactured by GIBCO), and this solution was stirred. This was adjusted into a pH of 7.4, and then the total amount thereof was set to 1000 ml. The resultant was filtrated with a 0.22 μm membrane filter and sterilized to prepare a Waymouth MB 752/1 culture medium solution.

The previously-prepared hepatocytes suspension was suspended into the prepared Waymouth MB 752/1 culture medium solution, and further the resultant suspension was inoculated onto the above-mentioned cell culture patterning substrate, which had the cell adhesion portions and the cell adhesion-inhibiting portions. This substrate was allowed to stand still in an incubator to which 5% CO₂ was supplied at 37° C. for 24 hours to cause the hepatocytes to adhere onto the entire surface of the substrate. This substrate was washed with PBS two times to remove non-adhering cells and dead cells. Thereafter, the culture medium solution was exchanged for a new culture medium solution.

While the exchange of the culture medium solution was repeated, the cells were continued to be cultured for 48 hours. The cells were then observed with an optical microscope. As a result, it was found out that the cells adhered along the cell adhesion portions of the cell culture patterning substrate.

### Example 2

### Formation of a Cell Culture Patterning Substrate

A quartz substrate, wherein a stripe-form shielding layer having light-shielding portions of 80 μm width and space portions of 300 μm width was formed on a surface of a base material, was prepared. A photocatalyst-containing layer and a cell adhesion-inhibiting layer were formed on the surface of this substrate with the shielding layer in the same way as in Example 1. Next, instead of using any photomask, ultraviolet rays were irradiated onto the entire back face of the substrate from the back face side of the substrate under the same conditions as in Example 1 to form a cell culture patterning substrate.

### (Cell Adhesion Process)

Cells were caused to adhere onto the above-mentioned patterning substrate in the same way as in Example 1. As a result, in this substrate also, it was found out that the cells adhered along the cell adhesion portions of the cell culture patterning substrate.

### Example 3

A hole of 14 mm diameter was made at the center of the bottom face of a commercially available plastic dish (manufactured by Corning Inc.) of 35 mm diameter. Subsequently, a glass substrate of about 0.1 mm thickness was used to form a cell culture patterning substrate in the same way as in Example 1. This cell culture patterning substrate was cut into a 21 mm square. Thereafter, the glass substrate of the cut cell culture patterning substrate was stuck onto the above-mentioned plastic dish through an adhesive agent, KE45T (Shin-Etsu Chemical Co., Ltd).

### (Cell Adhesion)

The plastic dish was sterilized with 70% ethanol, and washed with PBS. Thereafter, the dish was washed with a DMEM culture medium. Cells were then cultured in the same way as in Example 1. As a result, it was found out that the cells adhered along the cell adhesion portions in the plastic dish.

### Example 4

A quartz substrate with a shielding layer, about 0.1 mm in thickness, was used to form a cell culture patterning substrate having a cell adhesion-inhibiting layer in the same way as in Example 2. This cell culture patterning substrate was cut into a 21 mm square. Thereafter, in the same way as in Example 3, the quartz substrate of the cut cell culture patterning substrate was stuck onto the above-mentioned plastic dish.

### (Cell Culture)

The cells were cultured in the plastic dish in the same way as in Example 3. As a result, it was found out that the cells adhered along the cell adhesion portions in the plastic dish.

### Example 5

### Formation of a Patterning Substrate

A photocatalyst-containing layer was formed on a substrate in the same way as in Example 1. Subsequently, a solution of 5 g of isopropyl alcohol, 0.1 g of an organosilane, TSL8114 (manufactured by GE Toshiba Silicones), and 0.15 g of PEG-silane (Methoxypolyethylene glycol 5,000 trimethylsilyl ether, Fluka) was applied onto the photocatalyst-containing layer by spin coating. Thereafter, the substrate was heated at 150° C. for 10 minutes to form a cell adhesion-inhibiting layer. In this way, a patterning substrate was yielded.

### (Patterning of the Patterning Substrate, and Cell Adhesion)

The patterning substrate was patterned in the same way as in Example 1, so as to form a cell culture patterning substrate. Thereafter, cells were caused to adhere onto the cell culture patterning substrate in the same way as in Example 1. As a result, in the present examples also, it was found out that the cells adhered along the cell adhesion portions on the cell culture patterning substrate.

### Example 6

A cell culture patterning substrate was formed in the same way as in Example 5 except that a glass substrate of about 0.1 mm thickness was used. This was cut into a 21 mm square. Next, the glass substrate of the cut cell culture patterning substrate was stuck onto the above-mentioned plastic dish through the same steps as in Example 3.

### (Cell Culture)

Cells were cultured in the plastic dish in the same way as in Example 3. As a result, it was found out that cells adhered along the cell adhesion portions inside the plastic dish.

1. A coating liquid for patterning substrate, comprising a cell adhesion-inhibiting material which has cell adhesion-inhibiting material which has cell adhesion-inhibiting properties of inhibiting adhesion to cells and is decomposed or denatured by action of a photocatalyst on a basis of irradiation with energy, and a cell adhesive material which has cell adhesive properties at least after the material is irradiated with the energy.

2. The coating liquid for patterning substrate according to claim 1, wherein the cell adhesion-inhibiting material is a material having an oil repellency or a water repellency.

3. The coating liquid for patterning substrate according to claim 2, wherein the material having the oil repellency or the water repellency is organopolysiloxane that is a hydrolysis condensate or a cohydrolysis condensate of a silicon compound.

4. The coating liquid for patterning substrate according to claim 3, wherein the organopolysiloxane that is the hydrolysis condensate or the cohydrolysis condensate of the silicon compound is polysiloxane containing a fluoroalkyl group.

5. The coating liquid for patterning substrate according to claim 2, wherein the material having the oil repellency or the water repellency is organopolysiloxane in which a plurality of reactive silicones are crosslinked.
